(19) United States
(12) Patent Application Publication
(10) Pub. No.: US 2022/0096438 A1
(43) Pub. Date: Mar. 31, 2022

(54) METHODS OF TREATING MIGRAINE

(71) Applicant: Upsher-Smith Laboratories, LLC, Mamle Grove, MN (US)

(72) Inventors: Sagar Munjal, Skillman, NJ (US); Arun Jana, West Bengal (IN)

(21) Appl. No.: 17/273,811

(22) PCT Filed: Sep. 6, 2019
(86) PCT No.: PCT/IB2019/057539

§ 371 Date: Mar. 5, 2021

Related U.S. Application Data
(63) Continuation of application No. 16/125,337, filed on Sep. 7, 2018, now abandoned.

(51) Int. Cl. A61K 31/4045; A61K 9/00; A61P 25/06

(52) U.S. Cl. CPC A61K 31/4045; A61K 31/7028; A61P 25/06; A61K 9/0043

(57) ABSTRACT

The present application relates to a method of reducing a need of rescue medication and/or a need of more than one dose of migraine medication in a patient suffering from cephalic pain, including migraine, cluster headache, episodic migraine, or rapid escalating migraine. In some embodiments, the method includes administering intranasally a composition comprising sumatriptan, or a physiologically-acceptable salt or a solvate thereof, and an alkyl glycoside or a saccharide alkyl ester.

### TECHNICAL FIELD

The present application relates to a method of reducing a need of rescue medication and/or a need of more than one dose of migraine medication in a patient suffering from cephalic pain, including migraine, cluster headache, episodic migraine, or rapid escalating migraine.

### BACKGROUND

Sumatriptan is a selective 5-hydroxytryptamine ID (5-HT₁₀) receptor agonist useful for treatment of migraine. Sumatriptan has a chemical name 1-[3-(2-dimethylaminoethyl)-1H-indole-5-yl]-N-methyl-methane sulphonamide, which has the following structure (I).

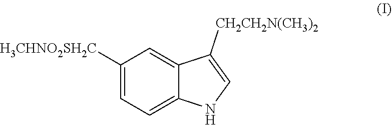

Broadly, all the triptans are selective 5HT receptor agonists useful for treatment of migraine.

U.S. Pat. No. 5,705,520 describes 3-[2-(dimethylamino) ethyl]-N-methyl-1H-methanesulphonamide sulphate salt (2:1) and pharmaceutically-acceptable solvates thereof, and pharmaceutical compositions containing the compound. The compound is useful in the treatment of conditions associated with cephalic pain, in particular migraine.

Many of the currently-available acute migraine treatments fail in providing sustained pain freedom for a period of 24 hours. Patients tend to take additional medication (rescue) and/or take second dose of same medication within the period of 24 hours, after taking the 1^{st} dose. Any acute treatment must be limited to a maximum of 2-3 days a week to prevent medication overuse headache and developing resistance or non-response to existing medications. Additionally, migraine-related functional disability, as well as the degree of patient satisfaction associated with its use, have not been assessed for available treatments. There is a long felt need to reduce the use of rescue medication and/or the need of second dose of migraine medication in a patient suffering from or susceptible to cephalic pain. It is also desired to have a migraine medication to provide pain freedom at 2 hours post-dose along with treatment satisfaction to patient population.

### SUMMARY

An embodiment of the present application relates to a method of reducing need of rescue medication use in a patient in need thereof, comprising intranasal administration of composition comprising sumatriptan, a physiologically-acceptable salt, or a solvate thereof, an alkyl glycoside or a saccharide alkyl ester and optionally at least one pharmaceutically-acceptable excipient.

In an aspect of the above embodiment, the need of rescue medication use in a patient is reduced by at least 50%.

In another aspect of the above embodiment, the need of rescue medication use in a patient is reduced by at least 50%, 45%, 40%, 35%, 30%, 25%, 20%, 15% or 10%.

In another embodiment, the present application relates to a method of reducing need of rescue medication use in a patient in need thereof, comprising intranasal administration of a composition comprising sumatriptan citrate or a physiologically-acceptable solvate thereof, an alkyl glycoside or a saccharide alkyl ester and at least one pharmaceutically-acceptable excipient.

In an aspect of the above embodiments, the rescue medication is selected from group comprising opioids, acetaminophen, aspirin, ibuprofen, rizatriptan, naratriptan, zolmitriptan, eletripan, almotriptan or dihydroergotamine.

Another embodiment of the present application relates to a method of reducing need of second dose of migraine medication in a patient in need thereof, comprising intranasal administration of composition comprising sumatriptan, a physiologically-acceptable salt, or a solvate thereof, an alkyl glycoside or a saccharide alkyl ester and optionally at least one pharmaceutically-acceptable excipient.

In an aspect of the above embodiment, the need of second dose of migraine medication is reduced by at least 30%.

In another aspect of the above embodiment, the need of second dose of migraine medication is reduced by at least 30%, 25%, 20%, 15% or 10%.

Another embodiment of the present application relates to a method of reducing need of second dose of migraine medication in a patient in need thereof, comprising intranasal administration of composition comprising sumatriptan citrate or a physiologically-acceptable solvate thereof, an alkyl glycoside or a saccharide alkyl ester and at least one pharmaceutically-acceptable excipient.

In an aspect of the above embodiments, the second dose of migraine medication comprises intranasal composition comprising sumatriptan, a physiologically-acceptable salt, or a solvate thereof, an alkyl glycoside or a saccharide alkyl ester and optionally at least one pharmaceutically-acceptable excipient.

Another embodiment of the present application relates to a method of reducing need of second dose of sumatriptan in a patient in need thereof, comprising intranasal administration of composition comprising sumatriptan citrate or a physiologically-acceptable solvate thereof, an alkyl glycoside or a saccharide alkyl ester and at least one pharmaceutically-acceptable excipient.

Another embodiment of the present application relates to a method of reducing need of rescue medication use or need of second dose of migraine medication in a patient in need thereof, comprising intranasal administration of composition comprising an aqueous solution of sumatriptan citrate or a physiologically-acceptable solvate thereof, an alkyl glycoside or saccharide alkyl ester, and optionally at least one pharmaceutically-acceptable excipient.

Another embodiment of the present invention includes a method of treating cephalic pain, which involves selecting a patient suffering from or susceptible to cephalic pain, who is identified as being susceptible to a need for rescue medication and/or more than one dose of abortive medication following onset of the cephalic pain; and administering intranasally a composition comprising sumatriptan, or a physiologically acceptable salt or a solvate thereof, and an alkyl glycoside or a saccharide alkyl ester.

In an aspect of the above embodiments, the composition upon intranasal administration provides at least one of the following pharmacokinetic parameters:
a. Tₘₐₓ value of less than or equal to about 30 minutes,
b. Cₘₐₓ ranges from about 14 to about 214 ng/mL,
c. AUC₀₋₂ ranging from about 22 to about 160 ng·h/mL,
d. AUC₀₋₆ ranging from about 25 to about 160 ng*h/mL, and/or
e. ratio of Cₘₐₓ to AUC_{0-inf} is at least about 0.3.

In another aspect of the above embodiments, said composition upon intranasal administration provides a Tₘₐₓ value of less than or equal to about 30 minutes or less than or equal to about 15 minutes.

In another aspect of the above embodiments, said composition provides a Tₘₐₓ substantially equivalent to subcutaneous sumatriptan injection comprising 4 mg or 6 mg sumatriptan base.

In another aspect of the above embodiments, the method comprises intranasal administration of a composition comprising an aqueous solution of sumatriptan, a physiologically-acceptable salt, or a solvate thereof, an alkyl glycoside or a saccharide alkyl ester, and optionally at least one pharmaceutically-acceptable excipient.

In another aspect of the above embodiments of the method, the composition, upon intranasal administration to said patient, provides at least one of the following clinical endpoints:
a. pain freedom in at least about 35% patient population at 2 hours post-dose;
b. pain relief in at least about 70% patient population at 2 hours post-dose;
c. sustained pain freedom in at least about 33% patient population from 2 to 24 hours post-dose; and
d. freedom from functional disability in at least about 45% patient population at 2 hours post-dose.

### BRIEF DESCRIPTION OF THE DRAWINGS

FIG. 1 shows a comparison of the average plasma levels of all patients at the various time points as indicated for the IMITREX® nasal spray reference and the phosphate salt of the sumatriptan pharmaceutical composition described herein.

FIG. 2 shows a comparison of the average plasma levels of all patients at the various time points as indicated for the IMITREX® nasal spray reference and the citrate salt of the sumatriptan pharmaceutical composition described herein.

FIG. 3 shows a comparison of the average plasma levels of all patients at the various time points as indicated for the IMITREX® nasal spray reference and the sulphate salt of the sumatriptan pharmaceutical composition described herein.

FIG. 4 shows a comparison of the average plasma concentration of all patients at various time points as indicated for the IMITREX® nasal spray reference and the sulphate salt of the sumatriptan (sulphate) upon intranasal administration of the pharmaceutical formulation comprising 0.1%, 0.15% & 2.0% concentrations of Permeation Enhancer (PE) respectively.

FIG. 5 shows the functional disability change at 2 hours post-dose (DB1) in patients acutely treating a migraine with moderate to severe pre-dose pain level for functional disability with the composition of Example 1 at 2 hours post-dose (−1.2) (n=48) compared with placebo (−0.6) (n=39).

FIG. 6 shows transformed scores for patients treated with the composition of Example 1 (n=37) compared with placebo (n=30) in connection with efficacy, function, and ease-of-use subscales at 24 hours post-dose.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom.

Unless otherwise defined, all technical and scientific terms used herein have the same meaning as commonly understood by one of ordinary skill in the art. In case of conflict, the specification of this document, including definitions, will control.

The terms “about,” “up to,” “generally,” and the like are to be construed as modifying a term or value such that it is not an absolute. Such terms will be defined by the circumstances and the terms that they modify, as those terms are understood by those of skill in the art. This includes, at the very least, the degree of expected experimental error, technical error, and instrumental error for a given experiment, technique, or an instrument used to measure a value. The term “about” is used to provide flexibility to a numerical range endpoint by providing that a given value may be “a little above” or “a little below” the endpoint. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum.

The term “commercially-available sumatriptan subcutaneous injection,” as used herein, refers to IMITREX® 6 mg injection (sumatriptan succinate, equivalent to 6 mg base/0.5 ml) or IMITREX STATDOSE® 4 mg injection (sumatriptan succinate, equivalent to 4 mg base/0.5 ml) or IMITREX STATDOSE® 6 mg injection (sumatriptan succinate, equivalent to 6 mg Base/0.5 ml) approved under NDA no. N020080 or its pharmaceutical equivalents or its therapeutic equivalents or later approved drugs which are designated as AB rated by US FDA as per Approved Drug Products with Therapeutic Equivalence Evaluations (34th edition) or drugs obtained marketing approval by US FDA through Abbreviated New Drug Application (ANDA) filing by establishing bioequivalence to such Product.

The term “pharmaceutically-acceptable excipient” is used to describe a substance which does not have inherent pharmacological activity and is used as an inactive ingredient.

The term “sumatriptan salt” is intended to include, but is not limited to, any of the following salts of sumatriptan: sulphate salt, citrate salt, phosphate salt, maleate salt, formate salt, and acetate salt. Other examples of salts of sumatriptan include, but are not limited to, hydrochloride, hydrobromide, nitrate, mesylate, benzoate, and succinate.

The term “sumatriptan salt” is also intended to include above defined salt of sumatriptan formed either externally or in situ during the preparation of the composition, by addition of the corresponding acid.

The term “citrate salt” indicates a source of citric acid that can be selected from the group containing citric acid anhydrous, citric acid monohydrate, trisodium citrate dihydrate, monosodium citrate, and citric acid solutions.

The term “cephalic pain,” as used herein, refers to any pain in the head such as cluster headache, chronic paroxysmal hemi-crania, headache associated with vascular disorders, headache associated with substances or their withdrawal (for example drug withdrawal), tension headache, and migraine.

In addition to “cephalic pain” experienced by a patient, patients typically suffer from other “most bothersome symptoms” (MBS), which are unique to the patient and based on a patient report that identifies such MBS, as is understood in the art. Such symptoms include, but are not necessarily limited to nausea, vomiting, photophobia (light sensitivity), and phonophobia (sound sensitivity). Other examples of such symptoms include, but are not limited to, sensitivity to smell, sensitivity to touch, blurred vision and other visual phenomena, light headedness, fainting, vertigo, dizziness, and mood changes.

The term “migraine,” as used herein, refers to migraine with or without aura.

The term “treating migraine,” as used herein, refers to treatment of acute migraine attacks with or without aura.

The term “treating cluster headache,” as used herein, refers to treatment of cluster headache episodes.

The term “subject,” as used herein, refers to a human who may or may not be suffering from cephalic pain.

The term “patient,” as used herein, refers to a human who is suffering from cephalic pain, such as migraine.

The term “rescue medication,” as used herein, refers to any medications, other than a study medication, or a composition comprising sumatriptan (or a physiologically-acceptable salt or solvate thereof), an alkyl glycoside or a saccharide alkyl ester, and optionally at least one pharmaceutically-acceptable excipient, taken to treat a migraine attack within the period of 2 hours to 24 hours after taking the first dose of a study medication or a composition comprising sumatriptan (or a physiologically-acceptable salt or solvate thereof), an alkyl glycoside or a saccharide alkyl ester, and optionally at least one pharmaceutically-acceptable excipient. Rescue medications typically include, but not limited to, opioids, acetaminophen, aspirin, ibuprofen, rizatriptan, naratriptan, zolmitriptan, eletripan, almotriptan, or dihydroergotamine.

The term “second dose of migraine medication,” as used herein, refers to administration of additional dose of a study medication or a composition comprising sumatriptan (or a physiologically-acceptable salt or solvate thereof), an alkyl glycoside or a saccharide alkyl ester, and optionally at least one pharmaceutically-acceptable excipient, taken to treat a migraine attack within the period of 2 hours to 24 hours after taking the first dose of study medication or the composition comprising sumatriptan (or a physiologically-acceptable salt or solvate thereof), an alkyl glycoside or a saccharide alkyl ester, and optionally at least one pharmaceutically-acceptable excipient.

Migraine pain intensity is rated on a 4 point ordinal scale where 0=no pain, 1=mild pain, 2=moderate pain, and 3=severe pain. The term “pain freedom,” as used herein, refers to a headache pain intensity rating of 0 (i.e., no pain). The term “pain relief,” as used herein, refers a reduction from pre-dose moderate or severe pain (2 or 3) to mild or none (0 or 1) post-dose.

The term “sustained pain freedom,” as used herein, refers to continued pain freedom at 24 hours post-dose, following pain freedom at 2 hours post-dose; and with no use of rescue medication or additional study medication, and no recurrence of headache pain within 2 to 24 hours post-dose.

The term “functional disability,” as used herein, refers to functional disability assessed on a scale of 1-3, where 0=no disability (able to function normally); 1=performance of daily activities mildly impaired (could still do everything but with difficulty); 2=performance of daily activities moderately impaired (unable to do some things); and 3=performance of daily activities severely impaired (unable to do most things, bed rest may have been necessary), wherein the function disability is assessed at pre-dose, 2 hours post-dose, and 24 hours post-dose.

An embodiment of the present application relates to a method of reducing the need for rescue medication in a patient following administration of an abortive medication for treatment of cephalic pain, such as a migraine medication for treatment of migraine. In the embodiment, the patient receives intranasal administration of a composition comprising sumatriptan, or a physiologically-acceptable salt or solvate thereof, an alkyl glycoside or a saccharide alkyl ester, and optionally at least one pharmaceutically-acceptable excipient.

Another embodiment of the present application relates to a method of reducing the need for rescue medication in a patient suffering from or susceptible to cephalic pain, such as migraine, cluster headache, episodic migraine, rapid escalating migraine, and the like.

Another embodiment of the present application relates to a method of reducing the need for rescue medication in a patient suffering from or susceptible to various types of migraine, including chronic migraine, migraine with aura, migraine without aura, migraine with allodynia, episodic migraine with allodynia, familial hemiplegic migraine, sporadic hemiplegic migraine, basilar-type migraine, status migrainosus, probable migraine, and retinal migraine.

In an aspect of the above embodiments, the need for rescue medication in the patient is reduced by at least about 50%.

In another aspect of the above embodiments, the need for rescue medication in the patient is reduced by at least about 40%.

In another aspect of the above embodiments, the need for rescue medication in the patient is reduced by at least about 30%.

In an aspect of the above embodiments, the need for rescue medication in the patient is reduced by at least about 50%, 45%, 40%, 35%, 30%, 25%, 20%, 15% or 10%.

In another aspect of the above embodiments, the rescue medication is selected from the group consisting of opioids, acetaminophen, aspirin, ibuprofen, rizatriptan, naratriptan, zolmitriptan, eletripan, almotriptan, and dihydroergotamine.

An embodiment of the present application relates to a method of reducing the need for a second dose of migraine medication in a patient after receiving a first dose of migraine medication, which involves the first dose being intranasally administered and including a composition comprising sumatriptan, a physiologically-acceptable salt or a solvate thereof, an alkyl glycoside or a saccharide alkyl ester, and optionally at least one pharmaceutically-acceptable excipient.

Another embodiment of the present application relates to a method of reducing the need for a second dose of migraine medication in a patient suffering from or susceptible to cephalic pain such as migraine or cluster headache or episodic migraine or rapid escalating migraine and the like.

Another embodiment of the present application relates to a method of reducing the need for a second dose of migraine medication in a patient suffering from or susceptible to various types of migraine including chronic migraine, migraine with aura, migraine without aura, migraine with allodynia, episodic migraine with allodynia, familial hemiplegic migraine, sporadic hemiplegic migraine, basilar-type migraine, status migrainosus, probable migraine, and retinal migraine.

In an aspect of the above embodiments, the need for a second dose of migraine medication in a patient is reduced by at least about 30%.

In another aspect of the above embodiments, the need for a second dose of migraine medication in a patient is reduced by at least about 20%.

In another aspect of the above embodiments, the need for a second dose of migraine medication in a patient is reduced by at least about 10%.

In an aspect of the above embodiments, the need for a second dose of migraine medication in a patient is reduced by at least about 30%, 25%, 20%, 15% or 10%.

In another aspect of the above embodiments, the second dose of migraine medication comprises an intranasally-administered composition comprising sumatriptan, a physiologically-acceptable salt or a solvate thereof, an alkyl glycoside or a saccharide alkyl ester, and optionally at least one pharmaceutically-acceptable excipient.

An embodiment of the present application relates to a method of reducing the need for rescue medication in a patient following administration of an abortive medication for treatment of cephalic pain, such as a migraine medication for treatment of migraine. In the embodiment, the patient receives intranasal administration of a composition comprising sumatriptan citrate or a physiologically-acceptable solvate thereof, an alkyl glycoside or a saccharide alkyl ester, and at least one pharmaceutically-acceptable excipient.

Another embodiment of the present application relates to a method of reducing the need for a second dose of migraine medication in a patient, comprising intranasally-administering a composition comprising sumatriptan citrate or a physiologically-acceptable solvate thereof, an alkyl glycoside or a saccharide alkyl ester, and at least one pharmaceutically-acceptable excipient.

Another embodiment of the present application relates to a method of reducing need for rescue medication or a second dose of migraine medication in a patient, comprising intranasally-administering a composition comprising an aqueous solution of sumatriptan citrate or a physiologically-acceptable solvate thereof, an alkyl glycoside or saccharide alkyl ester, and optionally at least one pharmaceutically-acceptable excipient.

Another embodiment of the present application relates to a method of reducing the need for a second dose of sumatriptan in a patient, comprising intranasally-administering a composition comprising sumatriptan citrate or a physiologically-acceptable solvate thereof, an alkyl glycoside or a saccharide alkyl ester, and at least one pharmaceutically-acceptable excipient.

Another embodiment of the present application relates to a method of reducing the need for rescue medication or a second dose of sumatriptan in a patient, comprising intranasally-administering a composition comprising an aqueous solution of sumatriptan citrate or a physiologically-acceptable solvate thereof, an alkyl glycoside or saccharide alkyl ester, and optionally at least one pharmaceutically-acceptable excipient.

In an aspect of the above embodiments, the intranasal composition comprises about 2.5 mg to about 25 mg of the sumatriptan base

In an aspect of the above embodiments, said alkyl glycoside or saccharide alkyl ester is present in a concentration of at least about 0.01%.

In another aspect of the above embodiments, said alkyl glycoside or saccharide alkyl ester is present in a concentration of from about 0.05 to about 2.5%.

In an aspect of the above embodiments, the composition, upon intranasal administration, provides a Tₘₐₓ value of less than 30 minutes upon said administration.

In another aspect of the above embodiments, the composition, upon intranasal administration, provides a Tₘₐₓ value of less than or equal to about 15 minutes.

In another aspect of the above embodiments, the composition, upon intranasal administration, provides a Tₘₐₓ substantially equivalent to subcutaneous sumatriptan injection comprising 4 mg or 6 mg sumatriptan base.

In another aspect of the above embodiments, the composition, upon intranasal administration, provides at least one of the following pharmacokinetic parameters:
a. Tₘₐₓ value of less than or equal to about 30 minutes,
b. Cₘₐₓ ranges from about 14 to about 214 ng/mL,
c. AUC₀₋₂ ranging from about 22 to about 160 ng*h/mL,
d. AUC₀₋₆ ranging from about 25 to about 160 ng*h/mL, and
e. ratio of Cₘₐₓ to AUC_{0-inf} is at least about 0.3.

In another aspect of the above embodiments, the composition, upon intranasal administration, provides at least one of the following clinical endpoints:
a. pain freedom in at least about 35% patient population at 2 hours post-dose;
b. pain relief in at least about 70% patient population at 2 hours post-dose;
c. sustained pain freedom in at least about 33% patient population from 2 to 24 hours post-dose; and
d. freedom from functional disability in at least about 45% patient population at 2 hours post-dose.

Another embodiment of the present application relates to a method of treating a patient suffering from or susceptible to cephalic pain, comprising intranasal administration of a composition comprising sumatriptan, a physiologically-acceptable salt or solvate thereof, an alkyl glycoside or a saccharide alkyl ester, and optionally at least one pharmaceutically-acceptable excipient.

Another embodiment of the present application relates to a method of treating a patient suffering from or susceptible to cephalic pain, such as migraine, cluster headache, episodic migraine, rapid escalating migraine, and the like, comprising intranasal administration of a composition comprising sumatriptan, a physiologically-acceptable salt or solvate thereof, an alkyl glycoside or a saccharide alkyl ester, and optionally at least one pharmaceutically-acceptable excipient.

Another embodiment of the present application relates to a method of treating a patient suffering from or susceptible to various types of migraine including chronic migraine, migraine with aura, migraine without aura, migraine with allodynia, episodic migraine with allodynia, familial hemiplegic migraine, sporadic hemiplegic migraine, basilar-type migraine, status migrainosus, probable migraine, and retinal migraine, comprising intranasal administration of a composition comprising sumatriptan, a physiologically-acceptable salt or solvate thereof, an alkyl glycoside or a saccharide alkyl ester, and optionally at least one pharmaceutically-acceptable excipient.

Another embodiment of the present application relates to a method of treating a patient suffering from or susceptible to cephalic pain, comprising intranasal administration of a composition comprising sumatriptan, a physiologically-acceptable salt or solvate thereof, an alkyl glycoside or a saccharide alkyl ester, and optionally at least one pharmaceutically-acceptable excipient, wherein said composition, upon intranasal administration to said patient, provides at least one of the following clinical endpoints:
(a) pain freedom in at least about 35% patient population at 2 hours post-dose;
(b) pain relief in at least about 70% patient population at 2 hours post-dose;
(c) sustained pain freedom in at least about 33% patient population from 2 to 24 hours post-dose; and
(d) freedom from functional disability in at least about 45% patient population at 2 hours post-dose.

Another embodiment of the present application relates to method for treating a patient suffering from or susceptible to cephalic pain, comprising intranasal administration of a composition comprising sumatriptan, a physiologically-acceptable salt, or a solvate thereof, an alkyl glycoside or a saccharide alkyl ester, and optionally at least one pharmaceutically-acceptable excipient, wherein said composition upon intranasal administration to said patient provides at least one of the following clinical endpoints:
(a) pain freedom in about 10% to about 50% patient population at 2 hours post-dose;
(b) pain relief in about 50% to about 90% patient population at 2 hours post-dose;
(c) sustained pain freedom in about 10% to about 45% patient population from 2 to 24 hours post-dose; and
(d) freedom from functional disability in about 20% to about 60% patient population at 2 hours post-dose.

In an aspect of the above embodiments, the present composition, upon intranasal administration, provides pain freedom in at least about 35% patient population at 2 hours post-dose.

In another aspect of the above embodiments, the present composition, upon intranasal administration, provides pain freedom in at least about 35%, 30%, 25%, 20%, 15% or 10% patient population at 2 hours post-dose.

In another aspect of the above embodiments, the present composition, upon intranasal administration, provides pain freedom in about 10% to about 50%, about 20% to about 50%, about 30% to about 50%, or about 40% to about 50% patient population at 2 hours post-dose.

In an aspect of the above embodiments, the present composition, upon intranasal administration, provides pain relief in at least about 70% patient population at 2 hours post-dose.

In another aspect of the above embodiments, the present composition, upon intranasal administration, provides pain relief in at least about 70%, 60% or 50% patient population at 2 hours post-dose.

In another aspect of the above embodiments, the present composition, upon intranasal administration, provides pain relief in about 50% to about 90%, about 60% to about 90%, about 70% to about 90%, or about 80% to about 90% patient population at 2 hours post-dose.

In an aspect of the above embodiments, the present composition, upon intranasal administration, provides sustained pain freedom in at least about 33% patient population from 2 to 24 hours post-dose.

In another aspect of the above embodiments, the present composition, upon intranasal administration, provides sustained pain freedom in at least about 33%, 30%, 25%, 20% or 15% patient population from 2 to 24 hours post-dose.

In another aspect of the above embodiments, the present composition, upon intranasal administration, provides sustained pain freedom in about 15% to about 45%, about 25% to about 45%, or about 35% to about 45% patient population from 2 to 24 hours post-dose.

In an aspect of the above embodiments, the present composition, upon intranasal administration, provides freedom from functional disability in at least about 45% patient population at 2 hours post-dose.

In another aspect of the above embodiments, the present composition, upon intranasal administration, provides freedom from functional disability in at least about 45%, 40%, 30% or 20% patient population at 2 hours post-dose.

In another aspect of the above embodiments, the present composition, upon intranasal administration, provides freedom from functional disability in about 20% to about 60%, about 30% to about 60%, about 40% to about 60%, or about 50% to about 60% patient population at 2 hours post-dose.

In an aspect of the above embodiments, the present composition, upon intranasal administration, provides absence of MBS in at least about 60% patient population at 2 hours post-dose.

In another aspect of the above embodiments, the present composition, upon intranasal administration, provides absence of MBS in at least about 60%, 55%, 50% or 45% patient population at 2 hours post-dose.

In another aspect of the above embodiments, the present composition, upon intranasal administration, provides absence of MBS in about 45% to about 75%, about 55% to about 75%, or about 65% to about 75% patient population at 2 hours post-dose.

In an aspect of the above embodiments, the present composition, upon intranasal administration, provides absence of nausea in at least about 65% patient population at 2 hours post-dose.

In another aspect of the above embodiments, the present composition, upon intranasal administration, provides absence of nausea in at least about 65%, 60% or 55% patient population at 2 hours post-dose.

In another aspect of the above embodiments, the present composition, upon intranasal administration, provides absence of nausea in about 55% to about 85%, about 65% to about 85%, about 75% to about 85%, or about 75% to about 80% patient population at 2 hours post-dose.

In an aspect of the above embodiments, the present composition, upon intranasal administration, provides absence of photophobia in at least about 60% patient population at 2 hours post-dose.

In another aspect of the above embodiments, the present composition, upon intranasal administration, provides absence of photophobia in at least about 60%, 55%, 50% or 45% patient population at 2 hours post-dose.

In another aspect of the above embodiments, the present composition, upon intranasal administration, provides absence of photophobia in about 45% to about 75%, about 55% to about 75%, about 65% to about 75%, or about 65% to about 70% patient population at 2 hours post-dose.

In an aspect of the above embodiments, the present composition, upon intranasal administration, provides absence of phonophobia in at least about 65% patient population at 2 hours post-dose.

In another aspect of the above embodiments, the present composition, upon intranasal administration, provides absence of phonophobia in at least about 65%, 60% or 55% patient population at 2 hours post-dose.

In another aspect of the above embodiments, the present composition, upon intranasal administration, provides absence of phonophobia in about 55% to about 85%, about 65% to about 85%, about 75% to about 85%, or about 75% to about 80% patient population at 2 hours post-dose.

In an aspect of the above embodiments, the present composition, upon intranasal administration, provides treatment satisfaction in at least about 60% patient population at 2 hours post-dose.

In another aspect of the above embodiments, the present composition, upon intranasal administration, provides treatment satisfaction in at least about 60%, 55%, 50%, 45% or 40% patient population at 2 hours post-dose.

In another aspect of the above embodiments, the present composition, upon intranasal administration, provides treatment satisfaction in about 40% to about 80%, about 50% to about 80%, about 60% to about 80%, or about 70% to about 80% patient population at 2 hours post-dose.

In an aspect of the above embodiments, the present composition, upon intranasal administration, provides treatment satisfaction for efficacy in at least about 55% patient population at 24 hours post-dose.

In another aspect of the above embodiments, the present composition, upon intranasal administration, provides treatment satisfaction for efficacy in at least about 50%, 45%, 40% or 35% patient population at 24 hours post-dose.

In another aspect of the above embodiments, the present composition, upon intranasal administration, provides treatment satisfaction for efficacy in about 35% to about 75%, about 45% to about 75%, about 55% to about 75%, about 65% to about 75%, or about 65% to about 70% patient population at 24 hours post-dose.

In an aspect of the above embodiments, the present composition, upon intranasal administration, provides treatment satisfaction for function in at least about 60% patient population at 24 hours post-dose.

In another aspect of the above embodiments, the present composition, upon intranasal administration, provides treatment satisfaction for function in at least about 60%, 55%, 50%, 45% or 40% patient population at 24 hours post-dose.

In another aspect of the above embodiments, the present composition, upon intranasal administration, provides treatment satisfaction for function in about 40% to about 80%, about 50% to about 80%, or about 60% to about 80% patient population at 24 hours post-dose.

Another embodiment of the present application relates to a method of reducing the need for rescue medication in a patient in need thereof, comprising intranasal administration of a composition comprising sumatriptan, or a physiologically-acceptable salt or solvate thereof, an alkyl glycoside or a saccharide alkyl ester, and optionally at least one pharmaceutically-acceptable excipient.

Another embodiment of the present application relates to a method of reducing the need for a second dose of migraine medication in a patient, comprising intranasal administration of a composition comprising sumatriptan, or a physiologically-acceptable salt or solvate thereof, an alkyl glycoside or a saccharide alkyl ester, and optionally at least one pharmaceutically-acceptable excipient.

Another embodiment of the present application relates to a method of reducing the need for rescue medication use or need of second dose of migraine medication in a patient, comprising intranasal administration of a composition comprising sumatriptan, or a physiologically-acceptable salt or solvate thereof, an alkyl glycoside or a saccharide alkyl ester, and optionally at least one pharmaceutically-acceptable excipient.

In an aspect of the above embodiments, the composition can be in the form of solution, suspension, emulsion, aerosol, powder, or the like.

In another aspect of the above embodiments, the salt of sumatriptan is selected from sulphate salt, citrate salt, phosphate salt, maleate salt, formate salt, or acetate salt of sumatriptan, or a physiologically-acceptable solvate thereof.

In another aspect of the above embodiments, the composition comprises an aqueous solution of sumatriptan, or a physiologically-acceptable salt or solvate thereof, and at least one pharmaceutically-acceptable excipient.

In another aspect of the above embodiments, the composition comprises sumatriptan, or a physiologically-acceptable salt or solvate thereof, and further comprises one or more pharmaceutically-acceptable excipients, such as a carrier, a flavor, an osmotic agent or a tonicity agent and/or a buffer.

In another aspect of the above embodiments, the composition comprises an aqueous solution of sumatriptan, or a physiologically-acceptable salt or solvate thereof, and further comprises one or more pharmaceutically-acceptable carriers, flavours, osmotic agents or tonicity agents, buffers, and/or excipients.

In another aspect of the above embodiments, the composition, upon intranasal administration, provides at least one of the following pharmacokinetic parameters:
a. Tₘₐₓ value of less than or equal to about 30 minutes,
b. Cₘₐₓ ranges from about 14 to about 214 ng/mL,
c. AUC₀₋₂ ranging from about 22 to about 160 ng*h/mL,
d. AUC₀₋₆ ranging from about 25 to about 160 ng*h/mL, and
e. ratio of Cₘₐₓ to AUC_{0-inf} is at least about 0.3.

In another aspect of the above embodiments, the composition, upon intranasal administration, provides a Tₘₐₓ value of less than or equal to about 15 minutes.

In another aspect of the above embodiments, the method comprises intranasal administration of a composition comprising sumatriptan, or a physiologically-acceptable salt or solvate thereof, an alkyl glycoside or a saccharide alkyl ester, and optionally at least one pharmaceutically-acceptable excipient.

In another aspect of the above embodiments, the method comprises intranasal administration of a composition comprising an aqueous solution of sumatriptan, or a physiologically-acceptable salt or solvate thereof, an alkyl glycoside or a saccharide alkyl ester, and optionally at least one pharmaceutically-acceptable excipient.

In an aspect of the above embodiments, the alkyl glycoside or the saccharide alkyl ester is selected from the group consisting of dodecyl maltoside, tridecyl maltoside, tetradecyl maltoside, octyl decyl alpha, and beta maltoside.

In an aspect of the above embodiments, said alkyl glycoside or saccharide alkyl ester is present in a concentration of at least about 0.01%.

In another aspect of the above embodiments, said alkyl glycoside or saccharide alkyl ester is present in a concentration of from about 0.02 to about 3.0%.

In another aspect of the above embodiments, said alkyl glycoside or saccharide alkyl ester is present in a concentration of from about 0.05 to about 2.5%.

In another aspect of the above embodiments, said alkyl glycoside or saccharide alkyl ester is present in a concentration of from about 0.1 to about 2.0%.

Suitable examples of the alkylglycosides or saccharide alkyl esters used in the compositions of the present application include, but are not limited to: alkyl glycosides, such as octyl-, nonyl-, decyl-, undecyl-, dodecyl-, tridecyl-, tetradecyl-, pentadecyl-, hexadecyl-, heptadecyl-, and octadecyl-[alpha]- or [beta]-D-maltoside, -glucoside or -sucroside (synthesized according to Koeltzow and Urfer; Anatrace Inc., Maumee, Ohio; Calbiochem, San Diego, Calif.; Fluka Chemie, Switzerland); alkyl thiomaltosides, such as heptyl, octyl, dodecyl-, tridecyl-, and tetradecyl-[beta]-D-thiomaltoside (synthesized according to Defaye, J. and Pederson, C, “Hydrogen Fluoride, Solvent and Reagent for Carbohydrate Conversion Technology” in Carbohydrates as Organic Raw Materials, 247-265 (F. W. Lichtenthaler, ed.) VCH Publishers, New York (1991); Ferenci, T., J. Bacteriol, 144:7-11 (1980)); alkyl thioglucosides, such as heptyl- or octyl 1-thio [alpha]- or [beta]-D-glucopyranoside (Anatrace, Inc., Maumee, Ohio; see Saito, S. and Tsuchiya, T. Chem. Pharm. Bull. 33:503-508 (1985)); alkyl thiosucroses (synthesized according to, for example, Binder, T. P. and Robyt, J. F., Carbohydr. Res. 140:9-20 (1985)); alkyl maltotriosides (synthesized according to Koeltzow and Urfer); long chain aliphatic carbonic acid amides of sucrose [beta]-amino-alkyl ethers; (synthesized according to Austrian Patent 382,381 (1987); derivatives of palatinose and isomaltamine linked by amide linkage to an alkyl chain (synthesized according to Kunz, M., “Sucrose-based Hydrophilic Building Blocks as Intermediates for the Synthesis of Surfactants and Polymers” in Carbohydrates as Organic Raw Materials, 127-153); derivatives of isomaltamine linked by urea to an alkyl chain (synthesized according to Kunz); long chain aliphatic carbonic acid ureides of sucrose [beta]-amino-alkyl ethers (synthesized according to Gruber and Greber, pp. 95-116); and long chain aliphatic carbonic acid amides of sucrose [beta]-amino-alkyl ethers (synthesized according to Austrian Patent No. 382,381 (1987), Chemical Abstracts, 108: 1 14719 (1988) and Gruber and Greber, pp. 95-116). Surfactants of the alkylglycoside and/or sucrose ester types have characteristic hydrophilic-lipophilic balance (HLB) numbers, which can be calculated or determined empirically (M. J. Schick, Nonionic Surfactants, Marcel Dekker, Inc., New York, p. 607, 1967). Examples of suitable penetration enhancers are also disclosed in US20060046962, US20060045869, US20060045868, US20090163447, US20090047347, US20080299079, US20080200418, US20060046969, which are incorporated herein by reference in their entirety and for the purpose stated.

In another aspect of above embodiments, the composition for intranasal administration has a pH in the range of about 4 to about 8.

Suitable examples of osmotic agents or tonicity agents used in the compositions using in the above embodiments include, but not limited to sodium chloride, potassium chloride, calcium chloride, dextrose, glucose, mannitol, and the like, or mixtures thereof.

The tonicity or osmolality of the compositions using in the above embodiments range from about 250 mOsmol/kg to about 750 mOsmol/kg such as from about 250 mOsmol/kg to about 350 mOsmol/kg.

Suitable examples of the buffers used in the compositions of the present application include, but not limited to, phosphate, borate, citrate, and other organic acids.

In another aspect, the present application provides a method of treating a human suffering from or susceptible to various types of migraine, including chronic migraine, migraine with aura, migraine without aura, migraine with allodynia, episodic migraine with allodynia, familial hemiplegic migraine, sporadic hemiplegic migraine, basilar-type migraine, status migrainosus, probable migraine, and retinal migraine.

In another aspect, the present application provides a method of treating a human suffering from or susceptible to migraine with allodynia, wherein said method comprises intranasal administration of a composition comprising sumatriptan, or a physiologically-acceptable salt or solvate thereof, and optionally at least one pharmaceutically-acceptable excipient, wherein said composition provides a Tₘₐₓ value of less than 30 minutes upon said administration.

In another aspect, the present application provides a method of treating a human suffering from or susceptible to episodic migraine with allodynia, wherein said method comprises intranasal administration of a composition comprising sumatriptan, or a physiologically-acceptable salt or solvate thereof, and optionally at least one pharmaceutically-acceptable excipient, wherein said composition provides a Tₘₐₓ value of less than 30 minutes upon said administration.

In another aspect, the present application provides a method of treating a human suffering from or susceptible to episodic migraine, wherein said method comprises intranasal administration of a composition comprising sumatriptan, or a physiologically-acceptable salt or solvate thereof, and optionally at least one pharmaceutically-acceptable excipient, wherein said composition provides a Tₘₐₓ value of less than 30 minutes upon said administration.

In another aspect the present application provides a method of treating a human suffering from or susceptible to episodic migraine, wherein said method comprises intranasal administration of a composition comprising sumatriptan, or a physiologically-acceptable salt or solvate thereof, an alkyl glycoside or a saccharide alkyl ester, and optionally at least one pharmaceutically-acceptable excipient, wherein said composition provides a Tₘₐₓ value of less than 30 minutes upon said administration.

Another aspect the present application provides a method of treating a human suffering from or susceptible to episodic migraine, comprising intranasal administration of a composition comprising sumatriptan, or a physiologically-acceptable salt or solvate thereof, an alkyl glycoside or a saccharide alkyl ester, and optionally at least one pharmaceutically-acceptable excipient, wherein upon said administration, said composition provides a Tₘₐₓ substantially equivalent to subcutaneous sumatriptan injection comprising 4 mg or 6 mg sumatriptan base.

In another aspect of the above embodiments, the composition, upon intranasal administration, provides at least one of the following pharmacokinetic parameters:
a. Tₘₐₓ value of less than or equal to about 30 minutes,
b. Cₘₐₓ ranges from about 14 to about 214 ng/mL,
c. AUC₀₋₂ ranging from about 22 to about 160 ng*h/mL,
d. AUC₀₋₆ ranging from about 25 to about 160 ng*h/mL, and
e. ratio of Cₘₐₓ to AUC_{0-inf} is at least about 0.3.

In another aspect of the above embodiments, the composition, upon intranasal administration, provides a Tₘₐₓ value of less than or equal to about 15 minutes.

The composition for intranasal administration required for a method of reducing the need for rescue medication or a second dose of migraine medication in a patient can be conveniently presented in a unit dose form. A unit dose for intranasal administration contains the sumatriptan in an amount of about 0.5 mg to 100 mg, or about 1 to 60 mg, or about 2 to 40 mg, which can be administered into either one or both nostrils.

An embodiment of the present application relates to a method making use of an intranasal composition comprising about 2.5 mg to about 25 mg of the sumatriptan base, such as 2.5 mg or 3 mg or 4 mg or 5 mg or 6 mg or 7 mg or 8 mg or 9 mg or 10 mg or 11 mg or 12 mg or 13 mg or 14 mg or 15 mg or 16 mg or 17 mg or 18 mg or 19 mg or 20 mg.

Another embodiment of the present application relates to a method making use of an intranasal composition administered using a single-use nasal spray device.

A unit dose formulation can be provided as a single dose in a sealed unit, for example, a vial of glass or plastic material, which can be filled and sealed using conventional manufacturing techniques. Alternatively, a sealed vial of plastic materials can be produced by form-fill-seal technology. In an embodiment, the vial and the components of the pharmaceutical composition filled therein are heat stable. The sealed vial can be sterilized, for example, by autoclaving at 120° C. for not less than 15 minutes to provide a sterile unit dosage vial, which can be assembled into a convenient delivery device prior to use. In another embodiment, the unit dose volume is 50 to 200 for example, 100 μl.

In an aspect of the above embodiments, the total volume of the unit dose administered is 25 μl, 75 μl, 100 μl, 125 μl, 150 μl, 175 μl or 200 μl.

The composition for intranasal administration required for an embodiment of the method of reducing need for rescue medication or a second dose of migraine medication in a patient can be provided in a liquid form or in the form of dry powder. The liquid form can be a solution applied directly to the nasal cavity by conventional means, for example, with a dropper, pipette, or a spray, or as a solution using pressurized metered-dose inhalers (pMDI), or as dry powders using dry powder inhaler devices (DPIs). Alternatively, the formulation can also be administered by breath actuated inhalers (BAIs). The dry powder form can be a spray-dried composition or a freeze dried composition having the drug in a micronized form and alternatively the drug can be in a microparticulate or a nanoparticulate form.

The present application is further illustrated by the following examples, which are provided merely to be exemplary of the invention described above and do not limit the scope of the application. Certain modifications and equivalents will be apparent to those skilled in the art and are intended to be included within the scope of the present application.

### EXAMPLES

### Example-1: Preparation of ‘Citrate Salt of Sumatriptan’ Composition

### Procedure:

Step-i: Preparation of phosphate buffer:
   0.2 grams of dibasic sodium phosphate & 10.0 grams of monobasic potassium phosphate were dissolved in sufficient water to produce 1000 mL.
Step-ii: Preparation of 1N Sodium hydroxide:
   1.0 gram of sodium hydroxide was dissolved in sufficient water to produce 25 mL.
Step-iii: Preparation of 5N citric acid monohydrate:
   70.04 grams of citric acid monohydrate was dissolved in sufficient quantity of purified water and the volume was made up with purified water to 200 mL.
Step-iv: The required quantity of citric acid monohydrate (5N) solution was added to the required quantity of water and mixed well to get a uniform mixture. The required quantity of sumatriptan was added to the mixture and it was stirred to dissolve the drug.
Step-v: The pH of solution obtained in Step-iv was checked and required quantity of sodium hydroxide (1N) was added to it to adjust the pH of the solution to 5.5±0.5.
Step-vi: A required quantity of phosphate buffer was added to the solution of Step-v and was mixed well. The pH was checked and was adjusted to the requisite value by adding a required quantity of sodium hydroxide if necessary (1N).
Step-vii: A required quantity of 1-O-n-Dodecyl-β-D-Maltopyranoside was added to a solution of Step-vi and mixed well till it dissolved completely. The volume was made up with phosphate buffer and the resulting solution was mixed well.
Step-viii: The solution of Step-vii was filtered through a membrane filter assembly.
Step-ix: The required quantity of solution of Step-viii was filled in a nasal spray device.

TABLE 1

| S. | | Qty/Unit dose (mg/100 μl) | | | |
|---|---|---|---|---|---|
| No. | Ingredient | For 5 mg | For 10 mg | For 15 mg | For 20 mg |
| 1 | Sumatriptan | 5.00 mg | 10.00 mg | 15.00 mg | 20.00 mg |
| 2 | Citric acid monohydrate * | 1.576 mg | 3.152 mg | 4.728 | 6.304 mg |
| 3 | 1-O-n-Dodecyl-β-D- | 0.20 mg | 0.20 mg | 0.20 mg | 0.20 mg |
| | Maltopyranoside | | | | |
| 4 | Sodium hydroxide | q.s. to adjust | q.s. to adjust | q.s. to adjust | q.s. to adjust |
| | | pH 5.0-6.0 | pH 5.0-6.0 | pH 5.0-6.0 | pH 5.0-6.0 |
| 5 | Purified water | 20.000 μl | 20.000 μl | 20.000 μl | 20.000 μl |
| 6 | Phosphate Buffer ** | q.s. to 100 μl | q.s. to 100 μl | q.s. to 100 μl | q.s. to 100 μl |

q.s.-quantum sufficient
* To be used as (5N) Citric acid solution in purified water.
** Contains Dibasic sodium phosphate and Monobasic potassium phosphate and to be mixed in the ratio of 1:50 in purified water for the preparation of phosphate buffer.

### Example-2: Preparation of ‘Phosphate Salt of Sumatriptan’ Composition

Pharmaceutical composition comprising phosphate salt of sumatriptan was prepared by similar procedure as described in Example-1, with the following quantities of the reacting materials.

TABLE 2

| Ingredient/Condition | Quantity/Value | |
|---|---|---|
| Sumatriptan | 20 | mg |
| 1-O-n-Dodecyl-β-D- | 0.2 | mg |
| Maltopyranoside | | |
| Phosphoric acid | 4.03 | μL |
| Citric acid monohydrate | — | |
| Sodium hydroxide | q.s. | |
| Water | 20 | μL |
| Phosphate buffer | q.s. to 100 | μL |

| | |
|---|---|
| pH | 6.5-7.5 |

### Examples 3-5: Preparation of ‘Formate, Acetate and Maleate Salts of Sumatriptan’ Composition

The compositions of formate, acetate and maleate salt of sumatriptan composition was prepared by following the procedure as described in Example-1 by taking appropriate quantities of the reacting substances and by maintaining appropriate suitable pH conditions.

TABLE 3

| | | Composition | Composition | Composition |
|---|---|---|---|---|
| S. | | of Example-3 | of Example-4 | of Example-5 |
| No | Ingredients | (Formate salt) | (Acetate salt) | (Maleate salt) |
| 1 | Sumatriptan | 20.00 mg | 20.00 mg | 20.00 mg |
| 2 | Formic acid * | 2.55 μl | NA | NA |
| 3 | Acetic acid ** | NA | 3.80 μl | NA |
| 4 | Maleic acid *** | NA | NA | 3.86 mg |
| 5 | 1-O-n-Dodecyl-β-D- | 0.2 mg | 0.2 mg | 0.2 mg |
| | Maltopyranoside | | | |
| 6 | Phosphate buffer | 100 μl | 100 μl | 100 μl |

| | | | |
|---|---|---|---|
| pH observed for final | 6.96 | 5.49 | 7.05 |
| solution | | | |

* To be used as (10N) Formic acid solution in water.
** To be used as (10N) Acetic acid solution in water.
*** To be used as (5N) Maleic acid solution in water.

### Example-6: Sumatriptan Citrate Salt Composition

Composition comprising sumatriptan citrate salt was prepared by following procedure:
Step-i: Preparation of phosphate buffer: 0.2 grams of dibasic sodium phosphate & 10.0 grams of monobasic potassium phosphate were dissolved in sufficient water to produce 1000 mL.
Step-ii: Preparation of 1N sodium hydroxide: 1.0 gram of sodium hydroxide was dissolved in required quantity of water and diluted with water to 25 mL.
Step-iii: The required quantity of sumatriptan-citric acid monohydrate complex was added to required quantity of water and mix well until it dissolves.
Step-iv: The pH of solution of step-iv was checked and required quantity of sodium hydroxide (1N) was added to it to adjust the pH of the solution to 5.5±0.5.
Step-v: The required quantity of phosphate buffer was added to solution of step-iv and mixed well. The pH was checked and required quantity of sodium hydroxide (1N) was added if necessary to adjust the pH of the solution to 5.5±0.5.
Step-vi: The required quantity of n-dodecyl-beta-D-maltopyranoside was added to solution of step-v and mixed well till it dissolved completely. The volume was made up with phosphate buffer and mixed well.
Step-vii: the solution of step-vi was filtered through a membrane filter assembly.

TABLE 4

| S. | | Qty/Unit dose |
|---|---|---|
| No. | Item Name | (Qty/100 μl) |
| 1 | Sumatriptan-Citric acid salt | 22.32 mg |
| 2 | n-Dodecyl-beta-D-maltopyranoside | 0.20 mg |
| 3 | Sodium hydroxide | q.s. to adjust pH 5.0-6.0 |
| 4 | Purified water | 20.00 μl |
| 5 | Phosphate buffer | q.s. to 100 μl |

### Example-7: Other Sumatriptan Salt Compositions

By taking appropriate sumatriptan salt, pharmaceutical compositions can be prepared by following the procedure as described in Example-6.

### Example-8: Clinical Study

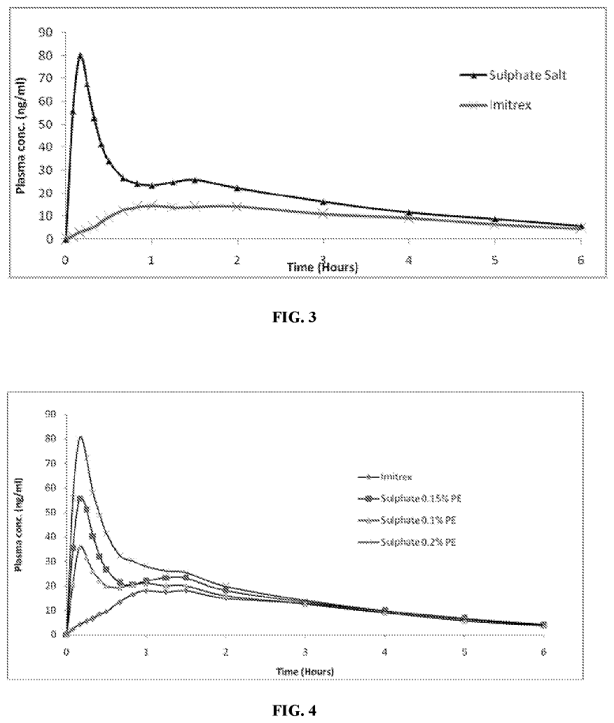

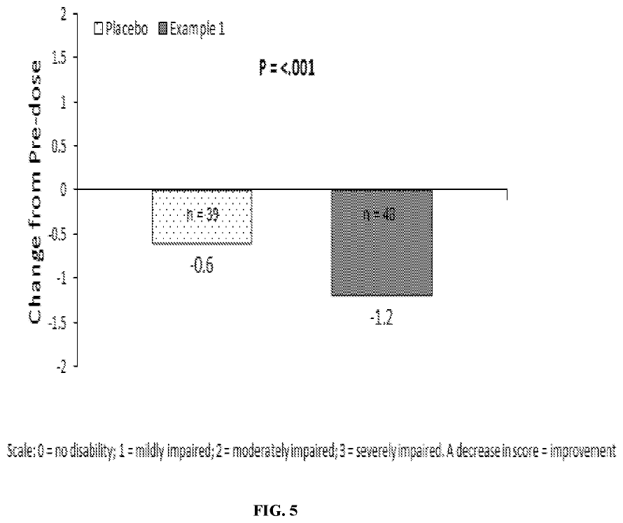

Study-1: Four-way crossover study of intranasal administration of sulphate salt of sumatriptan with Test-1, Test-2 and Test-3 having the permeation enhancer in concentrations of 0.1%, 0.15% and 0.2% respectively and IMITREX® 20 mg as the reference product (refer FIG. 4). Number of subjects completed study (n)=21).

Each drug solution (Test-1, Test-2, Test-3 and Reference) is administered to 24 patients in a three-way crossover study, with a washout period between doses of at least 7 days, as a 100 IA metered nasal spray using standard metered nasal spray devices such as those manufactured by Aptar Radolfzell GmbH, Germany, Valois Pharma, Le Neubourg, France, or Becton Dickinson, N.J., USA. Blood samples were collected from each patient at the timed intervals, for example 0.08, 0.17, 0.25, 0.33, 0.42, 0.6, 0.67, 0.83, 1, 2, 3, 4, 6, 8, 12 and 24 hours.

Sample for Test-1: Sulphate salt of sumatriptan composition 20 mg nasal spray (a final sumatriptan concentration of 20 mg per 100 μl spray and having about 0.1% of permeation enhancer (dodecyl maltoside)), manufactured by Dr. Reddy's Laboratories Ltd., India.

Sample for Test-2: Sulphate salt of sumatriptan composition 20 mg nasal spray (a final sumatriptan concentration of 20 mg per 100 μl spray and having about 0.15% of permeation enhancer (dodecyl maltoside)), manufactured by Dr. Reddy's Laboratories Ltd., India.

Sample for Test-3: Sulphate salt of sumatriptan composition 20 mg nasal spray (a final sumatriptan concentration of 20 mg per 100 μl spray and having about 0.2% of permeation enhancer (dodecyl maltoside)), manufactured by Dr. Reddy's Laboratories Ltd., India.

Reference: IMITREX® (sumatriptan) nasal spray, 20 mg per 100 μl), manufactured by GlaxoSmithKline.

### Example-9

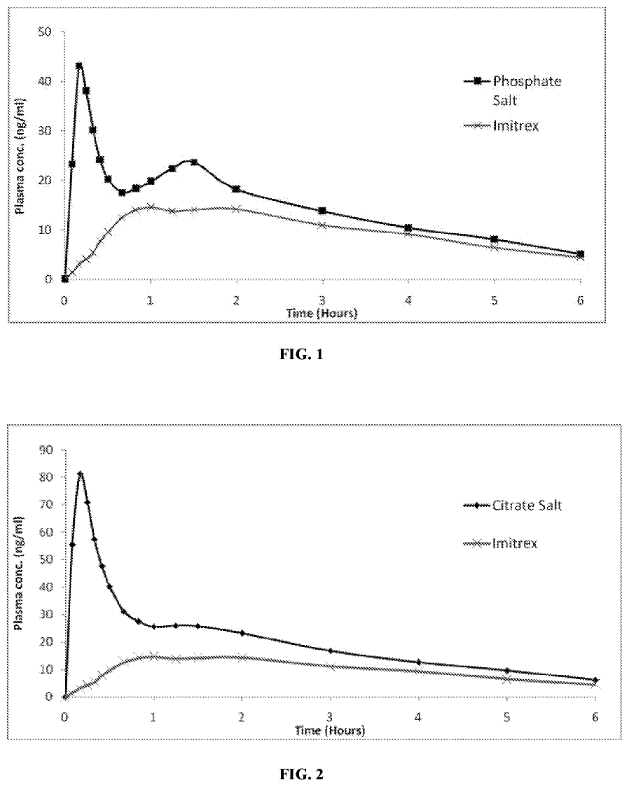

Study-2: Three-way crossover study of intranasal administration of various sumatriptan salts (phosphate, citrate and sulphate) having 0.2% of the permeation enhancer (refer FIG. 1, FIG. 2 and FIG. 3 respectively). Number of subjects completed study (n)=15.

Each drug solution (phosphate, citrate and sulphate salt of sumatriptan) is administered to 18 patients in a three-way crossover study, with a washout period between doses of at least 8 days, as a 100 μl metered nasal spray using standard metered nasal spray devices such as those manufactured by Aptar Radolfzell GmbH, Germany, Valois Pharma, Le Neubourg, France, or Becton Dickinson, N.J., USA. Blood samples were collected from each patient at the timed intervals, for example 0.08, 0.17, 0.25, 0.33, 0.42, 0.6, 0.67, 0.83, 1, 2, 3, 4, 6, 8, 12 and 24 hours.

Phosphate salt of sumatriptan composition 20 mg nasal spray (a final sumatriptan concentration of 20 mg per 100 μl spray and having about 0.2% of permeation enhancer (dodecyl maltoside)), manufactured by Dr. Reddy's Laboratories Ltd., India.

Citrate salt of sumatriptan composition 20 mg nasal spray (a final sumatriptan concentration of 20 mg per 100 μl spray and having about 0.2% of permeation enhancer (dodecyl maltoside)), manufactured by Dr. Reddy's Laboratories Ltd., India.

Sulphate salt of sumatriptan composition 20 mg nasal spray (a final sumatriptan concentration of 20 mg per 100 μl spray and having about 0.2% of permeation enhancer (dodecyl maltoside)), manufactured by Dr. Reddy's Laboratories Ltd., India.

### Example-10

Study-3: Three-way crossover study of intranasal administration of sulphate salt of sumatriptan with Test-1 having about 0.2% of permeation enhancer; Test-2 without permeation enhancer and IMITREX as the reference product. Number of subjects completed study (n)=15.

Each drug solution (Test-1, Test-2 and Reference samples) is administered to 18 patients in a three-way crossover study, with a washout period between doses of at least 4 days, as a 100 μl metered nasal spray using standard metered nasal spray devices such as those manufactured by Ing. Erich Pfeiffer GmbH, Radolfzell, Germany, Valois Pharma, Le Neubourg, France, or Becton Dickinson, N.J., USA. Blood samples were collected from each patient at the timed intervals, for example 0.08, 0.17, 0.25, 0.33, 0.42, 0.6, 0.67, 0.83, 1, 2, 3, 4, 6, 8, 12 and 24 hours.

Sample used in Test-1: Sulphate salt of sumatriptan composition 20 mg nasal spray (a final sumatriptan concentration of 20 mg per 100 μl spray and having about 0.2% of permeation enhancer (dodecyl maltoside)), manufactured by Dr. Reddy's Laboratories Ltd., India.

Sample used in Test-2: Sulphate salt of sumatriptan composition 20 mg nasal spray (a final sumatriptan concentration of 20 mg per 100 microliter spray and without permeation enhancer), manufactured by Dr. Reddy's Laboratories Ltd., India.

Reference: IMITREX® (sumatriptan) nasal spray, 20 mg per 100 μl), manufactured by GlaxoSmithKline.

TABLE 5
Area under the plasma concentration versus time curve
from time 0 to infinity (AUC_{0-inf})

| | AUC_{0-inf} (ng*h/mL) | | | | |
|---|---|---|---|---|---|
| | | Study 2 | | Study 2 | Study 2 |
| | Study 3 | (Sulphate | Study 1 | (Phosphate | (Citrate |
| Statistics | (0.2%) | salt) | (0.2%) | salt) | salt) |
| Arithmetic | 114.70 | 135.52 | 125.18 | 107.67 | 142.57 |
| Mean | | | | | |
| Standard | 52.98 | 48.78 | 46.72 | 25.03 | 45.83 |
| Deviation | | | | | |
| Maximum | 222.44 | 201.58 | 201.46 | 169.32 | 258.86 |
| Median | 109.04 | 130.73 | 117.94 | 108.22 | 136.85 |
| Minimum | 33.45 | 50.91 | 14.81 | 63.08 | 80.16 |
| Geometric | 101.68 | 125.64 | 112.78 | 104.98 | 136.27 |
| Mean | | | | | |
| CV % | 46.19 | 36.0 | 37.33 | 23.25 | 32.15 |

TABLE 6
Peak plasma concentration for sumatriptan (Cₘₐₓ)

| | Cₘₐₓ (ng/mL) | | | | |
|---|---|---|---|---|---|
| | | Study 2 | | Study 2 | Study 2 |
| | Study 3 | (Sulphate | Study 1 | (Phosphate | (Citrate |
| Statistics | (0.2%) | salt) | (0.2%) | salt) | salt) |
| Arithmetic | 60.50 | 84.98 | 83.69 | 44.78 | 85.30 |
| Mean | | | | | |
| Standard | 31.93 | 57.79 | 46.44 | 17.18 | 34.56 |
| Deviation | | | | | |
| Maximum | 110.88 | 213.11 | 173.43 | 81.22 | 186.33 |
| Median | 67.48 | 85.30 | 78.35 | 40.26 | 86.65 |
| Minimum | 8.44 | 15.62 | 5.95 | 22.69 | 48.62 |
| Geometric | 48.65 | 67.68 | 67.54 | 41.96 | 79.95 |
| Mean | | | | | |
| CV % | 55.05 | 68.01 | 55.50 | 38.37 | 40.52 |

TABLE 7
Time to peak plasma concentration for sumatriptan (Tₘₐₓ)

| | Tₘₐₓ (h) | | | | |
|---|---|---|---|---|---|
| | | Study 2 | | Study 2 | Study 2 |
| | Study 3 | (sulphate | Study 1 | (Phosphate | (Citrate |
| Statistics | (0.2%) | salt) | (0.2%) | salt) | salt) |
| Arithmetic | 0.21 | 0.24 | 0.21 | 0.43 | 0.161 |
| Mean | | | | | |
| Standard | 0.13 | 0.28 | 0.18 | 0.55 | 0.05 |
| Deviation | | | | | |
| Maximum | 0.66 | 1.25 | 1.00 | 0.25 | 0.25 |
| Median | 0.167 | 0.167 | 0.167 | 0.167 | 0.167 |
| Minimum | 0.083 | 0.083 | 0.083 | 0.083 | 0.083 |
| CV % | 62.97 | 114.74 | 84.84 | 127.76 | 30.76 |

TABLE 8
Area under the plasma concentration versus
time curve from time 0 to Tₘₐₓ (AUC_{0-Tmax})

| | AUC_{0-Tmax} (ng · h/mL) | | | | | |
|---|---|---|---|---|---|---|
| | | Study 2 | | Study 2 | Study 2 | Study 3 |
| | Study 3 | (Sulphate | Study 1 | (Phosphate | (Citrate | (IMITREX |
| Statistics | (0.2%) | salt) | (0.2%) | salt) | salt) | 20 mg) |
| Geometric Mean | 4.51 | 5.99 | 6.22 | 3.39 | 12.99 | 13.54 |
| Arithmetic Mean | 6.05 | 7.93 | 7.95 | 3.72 | 13.63 | 14.36 |
| 95% CI | 4.3-7.8 | 4.9-11.0 | 6.0-9.9 | 3.0-4.5 | 11.6-15.7 | 12.2-16.5 |

TABLE 9
Ratio of Cₘₐₓ to AUC_{0-inf} (Cₘₐₓ/AUC_{0-inf})

| | Cₘₐₓ/AUC_{0-inf} | | | | |
|---|---|---|---|---|---|
| | | Study 2 | | Study 2 | Study 2 |
| | Study 3 | (Sulphate | Study 1 | (Phosphate | (Citrate |
| Statistics | (0.2%) | salt) | (0.2%) | salt) | salt) |
| Geometric | 0.47 | 0.53 | 0.59 | 0.18 | 0.47 |
| Mean | | | | | |
| Arithmetic | 0.52 | 0.62 | 0.66 | 0.41 | 0.59 |
| Mean | | | | | |
| 95% CI | 0.5-0.54 | 0.5-0.71 | 0.6-0.71 | 0.37-0.44 | 0.57-0.62 |

TABLE 10
Area under the plasma concentration versus time
curve from time 0 to 2 hours (AUC₀₋₂)

| | AUC₀₋₂ (ng · h/mL) | | |
|---|---|---|---|
| | Study 2 | Study 2 | Study 2 |
| | (Sulphate | (Phosphate | (Citrate |
| Statistics | salt) | salt) | salt) |
| Arithmetic | 63.81 | 44.97 | 68.07 |
| Mean | | | |
| Standard | 27.92 | 11.11 | 29.59 |
| Deviation | | | |
| Maximum | 116.49 | 60.80 | 156.13 |
| Median | 69.08 | 46.57 | 61.11 |
| Minimum | 22.05 | 25.79 | 38.39 |
| Geometric | 57.16 | 43.58 | 63.49 |
| Mean | | | |
| CV % | 43.75 | 24.72 | 43.48 |

TABLE 11
Area under the plasma concentration versus time
curve from time 0 to 6 hours (AUC₀₋₆)

| | AUC₀₋₆ (ng · h/mL) | | |
|---|---|---|---|
| | Study 2 | Study 2 | Study 2 |
| | (Sulphate | (Phosphate | (Citrate |
| Statistics | salt) | salt) | salt) |
| Arithmetic | 68.08 | 88.98 | 44.97 |
| Mean | | | |
| Standard | 29.60 | 21.87 | 11.12 |
| Deviation | | | |
| Maximum | 156.13 | 142.27 | 60.80 |
| Median | 61.12 | 91.46 | 46.58 |
| Minimum | 38.39 | 55.32 | 25.79 |
| Geometric | 63.50 | 86.50 | 43.59 |
| Mean | | | |
| CV % | 43.48 | 24.58 | 24.72 |

### Example 11

The need of rescue medication after administration of intranasal administration of Example 1 (sumatriptan 10 mg) is extracted from a multi-center, randomized, double-blind, placebo-controlled efficacy and safety study in episodic migraine with or without aura. Additionally, proportion of patients with pain freedom and pain relief at 2 hours post-dose, absence of MBS and associated symptoms and sustained pain freedom from 2 to 24 hours post-dose were also assessed.

Treatments:

Example 1 and placebo were separately provided in a single-use nasal spray device calibrated to deliver 100 μl spray which was self-administered intra-nasally once at the onset of acute migraine pain during each acute migraine episode.

Method:

If a patient did not experience sufficient relief with the first dose of the study medication, he/she may have taken a second dose of study medication or rescue medication after 2 hours or more after taking the first dose of study medication and only after completing the 2 hour post-dose assessments in the eDiary.

If a patient did not experience any relief at all from the first dose of study medication after 2 hours, he/she should not have taken a second dose but may have taken rescue medication instead, if needed. Technically, rescue medications were any medications taken to treat a migraine attack between 2 and 24 hours after taking study medication. Rescue medication included non-steroidal anti-inflammatory drugs or other migraine medications like opioids, acetaminophen, aspirin, ibuprofen, rizatriptan, naratriptan, zolmitriptan, eletripan, almotriptan or dihydroergotamine, prescription or non-prescription drugs, vitamins, herbal, and dietary supplements (including St John's Wort).

After patients completed pre-dosing assessments and then reported that they dosed with the study medication in the eDiary, outcome measures were assessed at 10, 15, 20, 30, 60, 90, and 120 minutes, as well as at 24 hours, post-dose. Migraine pain intensity was rated on a 4 point ordinal scale where 0=no pain, 1=mild pain, 2=moderate pain, and 3=severe pain. The presence of symptoms of nausea, photophobia, and phonophobia were assessed as Yes (present) or No (not present). The MBS was selected before dosing by patients who reported the presence of more than one pre-dose symptoms. If only one symptom was present pre-dose, it was considered the MBS.

The assessments were for the proportion of subjects with migraine pain-freedom; pain relief; sustained pain freedom (2-24 hours post-dose); freedom from the MBS; and freedom from nausea, photophobia, and phonophobia, and they were each assessed at post-dose time points (10, 15, 20, 30, 60, and 90 minutes).

Results:

A significantly lower percentage of Example-1 patients used rescue medication—75%, and additional/second dose of study medication within 2 to 24 hours post dose—50% compared with placebo patients. Additionally, significantly higher percentage of Example-1 patients had pain freedom at 2 hours post-dose—48.7%, pain relief at 2 hours post-dose—34%, absence of MBS at 2 hours post-dose—44.2%, freedom from associated symptoms (nausea—46.2%, photophobia—46%, phonophobia—48.8%) and sustained pain freedom for 2 to 24 hours post-dose—64.5%, compared to placebo. The observations are listed in table nos. 12-13.

TABLE 12

| | | | | |
|---|---|---|---|---|
| Sr. | | Placebo | Example-1 | |
| No. | Study parameters | N = 43 | N = 50 | Results |

% reduction in need of rescue medication use and/or need of
second dose medication compared to placebo

| 1. | Number of patients with | 41 | 49 | — |
|---|---|---|---|---|
| | non-missing assessments | | | |
| | for use of second study | | | |
| | medication dose and rescue | | | |
| | medication criteria[s] | | | |
| 2. | Number of patients with | 20 | 6 | — |
| | use of second study | | | |
| | medication dose or rescue | | | |
| | medication within 2 to 24 | | | |
| | hours post-dose | | | |
| 3. | Proportion (%) (95% CI) [a] | 48.8 | 12.2 | 75% |
| | | (32.9, 64.9) | (4.6, 24.8) | |
| 4. | P -value | <0.001 | | |
| 5. | Use of second study | 6 | 3 | 50% |
| | medication dose within | (14.0) | (6.0) | |
| | 2 to 24 hours post-dose, | | | |
| | n (%) | | | |
| 6. | Use of rescue medication | 17 | 5 | 74.68% |
| | within 2 to 24 hours | (39.5) | (10.0) | |
| | post-dose, n (%) | | | |

Abbreviations: CI = confidence interval; DB1 = first double-blind treatment period;
Note:
P-values were obtained from Fisher's exact test
Note:
n is the number of patients who met the indicated criterion.
Note:
Percentages were calculated using N as the denominator.
[a] Proportion (%) was based on number of patients with non-missing assessment

TABLE 13

| | | | |
|---|---|---|---|
| Sr. | | Placebo | Example-1 |
| No. | Study parameters | N = 43 | N = 50 |

Pain freedom at 2 hours post-dose compared to placebo

| | | | |
|---|---|---|---|
| 1. | Proportion (%) (95% CI) [a] | 22.5 | 43.8 |
| | | (10.8, 38.5) | (29.5 58.8) |

| | | |
|---|---|---|
| 2. | P-value | 0.004 |

Pain relief at 2 hours post-dose compared to placebo

| | | | |
|---|---|---|---|
| 1. | Proportion (%) (95% CI) [a] | 55.0 | 83.3 |
| | | (38.5, 70.7) | (69.8, 92.5) |

| | | |
|---|---|---|
| 2. | P-value | 0.005 |

Absence of MBS at 2 hours post-dose compared to placebo

| | | | |
|---|---|---|---|
| 1. | Proportion (%) (95% CI) [a] | 39.5 | 70.7 |
| | | (24.0, 56.6) | (54.5, 83.9) |

| | | |
|---|---|---|
| 2. | P-value | 0.007 |

Freedom from associated symptoms
Nausea

| | | | |
|---|---|---|---|
| 1. | Proportion (%) (95% CI) [a] | 42.1 | 78.3 |
| | | (20.3, 66.5) | (56.3, 92.5) |

| | | |
|---|---|---|
| 2. | P-value | 0.026 |

Photophobia

| | | | |
|---|---|---|---|
| 1. | Proportion (%) (95% CI) [a] | 38.9 | 71.8 |
| | | (23.1, 56.5) | (55.1, 85.0) |

| | | |
|---|---|---|
| 2. | P-value | 0.05 |

Phonophobia

| | | | |
|---|---|---|---|
| 1. | Proportion (%) (95% CI) [a] | 40.0 | 78.1 |
| | | (22.7, 59.4) | (60.0, 90.7) |

| | | |
|---|---|---|
| 2. | P-value | 0.04 |

Sustained pain freedom for 2 to 24 hours post-dose

| | | | |
|---|---|---|---|
| 1. | Proportion (%) (95% CI) [a] | 13.8 | 38.9 |
| | | (3.9, 31.7) | (23.1, 56.5) |

| | | |
|---|---|---|
| 2. | P-value | 0.029 |

### Example 12

Patients were randomized in a double-blinded fashion to treat one attack (DB1) with composition of Example-1 or placebo within one hour of experiencing moderate to severe migraine pain; The endpoint was the functional disability, change at 2 hour post-dose (DB1) compared between composition of example-1 and placebo and treatment satisfaction at 2 hours post-dose (DB1) compared between composition of example 1 and placebo. Patients reported data in real-time eDiary.

Treatment satisfaction and functional disability was assessed using a validated Patient Perception of Migraine Questionnaire-Revised [PPMQ-R]; patients were evaluated at baseline, at pre-dose, 2 hour and 24 hour post-dose.

Result:

Functional Disability, change at 2 hour post-dose (DB1):

In patients acutely treating a migraine with moderate to severe pre-dose pain level, mean scores for functional disability significantly improved (p<0.001) for composition of example-1 at 2 hour post-dose (−1.2) (n=48) compared with placebo (−0.6) (n=39). See FIG. 5.

Treatment Satisfaction at 2 hours post-dose (DB1):

In accord, the overall satisfaction item was significantly better (p=0.013) for composition of example-1 at 2 hour post-dose (n=48) compared with their usual migraine medication assessed at baseline (n=48). See table no. 14 given below.

TABLE 14
Treatment satisfaction at 2 hours post-dose

| | | | p value, |
|---|---|---|---|
| | | | Example-1 |
| Study Parameters | Placebo | Example-1 | vs. Placebo |
| Satisfaction with usual | 41 | 48 | — |
| migraine medication | | | |
| (Baseline) | | | |
| Mean (SD) | 3.0 (1.4) | 3.6 (1.6) | — |
| Median (Min, Max) | 3.0 (1, 6) | 4.0 (1, 7) | — |
| Satisfaction with study | 38 | 48 | — |
| medication post-dose | | | |
| Mean (SD) | 4.1 (2.1) | 2.8 (1.5) | .003 |
| Median (Min, Max) | 4.0 (1, 7) | 2.0 (1, 6) | — |
| Difference from Baseline | — | −0.8 | — |
| p value, Example no 1 | — | .013 | — |
| post-dose versus Baseline | | | |

PPMQ-R, subscale & total, baseline vs. example-1 (24 hr Post-dose; DB1):

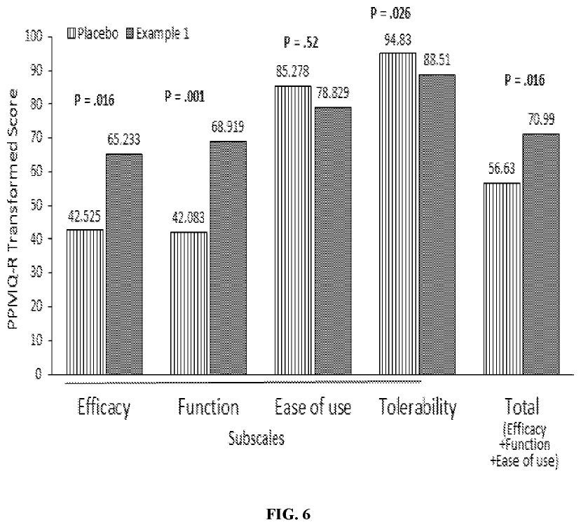

Patients treated with composition of example-1 (n=37) had significantly higher satisfaction compared with placebo (n=30) for the efficacy subscale (mean score 65.23 versus 42.53; p=0.016) and the function subscale (mean score 68.92 versus 42.08; p=0.001), but no significant difference was observed for ease-of-use subscale (mean score 78.83 versus 85.28; p=0.520) at 24 hours post-dose. See FIG. 6.

The Total Score (Efficacy+Function+Ease of use) was significantly better for composition of example-1 compared with placebo (mean score 71.0 versus 56.63; p=0.016).

PPMQ-R Global Items (DB1):

The Global items: medication effectiveness and overall satisfaction were significantly better for composition of example 1 at 24 hours (p=0.027 and p=0.019, respectively). See table no. 15 given below.

TABLE 15
PPMQ-R Global Items

| | |
|---|---|
| Placebo | DFN-02 |
| N = 43 | N = 50 |

Global item

| | | |
|---|---|---|
| Medication effectiveness | | |
| n | 30 | 37 |
| Mean (SD) | 4.4 (2.39) | 3.1 (1.92) |
| Median | 4.0 | 2.0 |
| Min, Max | 1, 7 | 1, 7 |
| P-value | | 0.027 |
| Overall satisfaction | | |
| n | 30 | 37 |
| Mean (SD) | 4.3 (2.31) | 3.0 (1.94) |
| Median | 4.0 | 2.0 |
| Min, Max | 1, 7 | 1, 7 |
| P-value | — | 0.019 |

Results:

Patients were also more likely to be satisfied or very satisfied than placebo-treated subjects at 2 hours post-dose (70.0% vs 44.2%, P=0.027). At 24 hours post-dose, mean scores were significantly superior to placebo on the PPMQ-R subscales for efficacy (65.2 vs 42.5, P=0.016) and function (68.9 vs 42.1, P=0.001), as well as the total score (71.0 vs 56.6, P=0.016), with no difference in ease of use. The observations are listed in table no. 17.

TABLE 17

| | | | |
|---|---|---|---|
| | | Placebo | Example-1 |
| Sr. No. | Study parameters | N = 43 | N = 50 |

No functional disability at 2 hours post-dose compared to placebo

| | | | |
|---|---|---|---|
| 1. | Proportion (%) | 28.2 | 56.3 |

| | | |
|---|---|---|
| 2. | P-value | 0.010 |

Mild or no functional disability at 2 hours post-dose compared to placebo

| | | | |
|---|---|---|---|
| 1. | Proportion (%) | 64.1 | 83.3 |

| | | |
|---|---|---|
| 2. | P-value | 0.050 |

Treatment satisfaction at 2 hours post-dose compared to placebo

| | | | |
|---|---|---|---|
| 1. | Proportion (%) | 44.2 | 70 |

| | | |
|---|---|---|
| 2. | P-value | 0.027 |

Treatment satisfaction for efficacy at 24 hours post-dose compared to placebo

| | | | |
|---|---|---|---|
| 1. | Proportion (%) | 42.5 | 65.2 |

| | | |
|---|---|---|
| 2. | P-value | 0.016 |

Treatment satisfaction for function at 24 hours post-dose compared to placebo

| | | | |
|---|---|---|---|
| 1. | Proportion (%) | 42.1 | 68.9 |

| | | |
|---|---|---|
| 2. | P-value | 0.001 |

Treatment satisfaction for total score (efficacy + function + ease of use) at
24 hours post-dose compared to placebo-with no difference in ease of use

| | | | |
|---|---|---|---|
| 1. | Proportion | 56.6 | 71.0 |

| | | |
|---|---|---|
| 2. | P-value | 0.016 |

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims), are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., a system having at least one of A, B, and C″ would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., a system having at least one of A, B, or C″ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms or both terms. For example, the phrase “A or B” will be understood to include the possibilities of “A” or “B” or “A and B.”

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

What is claimed is:

1. A method of treating cephalic pain, comprising:
selecting a patient suffering from or susceptible to cephalic pain, who is identified as being susceptible to a need for rescue medication and/or more than one dose of abortive medication following onset of the cephalic pain; and
administering intranasally a composition comprising sumatriptan, or a physiologically-acceptable salt or a solvate thereof, and an alkyl glycoside or a saccharide alkyl ester.

2. The method of claim 1, wherein said composition further includes at least one pharmaceutically-acceptable excipient.

3. The method of claim 1, wherein said composition, upon intranasal administration to said patient, provides at least one of the following clinical endpoints:
(a) pain freedom in at least about 35% patient population at 2 hours post-dose;
(b) pain relief in at least about 70% patient population at 2 hours post-dose;
(c) sustained pain freedom in at least about 33% patient population from 2 to 24 hours post-dose; and
(d) freedom from functional disability in at least about 45% patient population at 2 hours post-dose.

4. The method of claim 1, wherein said composition comprises sumatriptan in an amount of about 2.5 mg to about 25 mg of sumatriptan base.

5. The method of claim 1, wherein said alkyl glycoside or saccharide alkyl ester is present in an amount of from about 0.05% to about 3.0%.

6. The method of claim 5, wherein said alkyl glycoside or saccharide alkyl ester is selected from the group consisting of: dodecyl maltoside (1-O-n-dodecyl-β-D-maltopyranoside), tridecyl maltoside, sucrose monododecanoate, sucrose monotridecanoate, and sucrose monotetradecanoate.

7. The method of claim 1, wherein said salt of sumatriptan is selected from group consisting of: sulphate salt, citrate salt, phosphate salt, maleate salt, formate salt, and acetate salt.

8. The method of claim 1, wherein said composition, upon intranasal administration, provides absence of nausea in at least about 65% of patients at 2 hours post-dose.

9. The method of claim 1, wherein said composition, upon intranasal administration, provides an absence of photophobia in at least about 60% of patients at 2 hours post-dose.

10. The method of claim 1, wherein said composition, upon intranasal administration, provides an absence of phonophobia in at least about 65% patient population at 2 hours post-dose.

11. The method of claim 1, wherein said composition, upon intranasal administration, provides treatment satisfaction in at least about 60% of patients at 2 hours post-dose.

12. The method of claim 1, wherein said composition is in the form of a solution, suspension, emulsion, aerosol, or powder.

13. The method of claim 1, wherein the cephalic pain is migraine.

14. The method of claim 13, wherein the patient is susceptible to the need for rescue medication, and such need is reduced by at least about 50%.

15. The method of claim 1, wherein said rescue medication is selected from group comprising opioids, acetaminophen, aspirin, ibuprofen, rizatriptan, naratriptan, zolmitriptan, eletripan, almotriptan or dihydroergotamine.

16. The method of claim 1, wherein said composition provides a Tₘₐₓ value less than or equal to about 30 minutes.

17. The method of claim 1, wherein said composition, upon intranasal administration, provides a Tₘₐₓ substantially equivalent to that of a subcutaneous sumatriptan injection comprising 4 mg or 6 mg sumatriptan base.

18. The method of claim 13, wherein the patient is susceptible to the need for more than one dose of abortive medication, wherein the abortive medication is migraine medication, and wherein the need for a second dose of migraine medication is reduced by at least about 30%.

19. The method of claim 1, wherein the cephalic pain is migraine, cluster headache, episodic migraine, or rapid escalating migraine.
